(19) United States
(12) Patent Application Publication
(10) Pub. No.: US 2026/0315376 A1
(43) Pub. Date: Oct. 15, 2026

(54) NOVEL OPHTHALMIC COMPOSITION

(71) Applicant: Cipla Limited, Mumbai (IN)

(72) Inventors: Arunprasath KALIAPERUMAL, Weston, FL (US); Preeti Prashant RAUT, Mumbai (IN); Trupti CHITRE, Mumbai (IN); Vinayak MARATHE, Mumbai (IN); Rahul NARKHEDE, Nashik (IN)

(21) Appl. No.: 19/137,433

(22) PCT Filed: Nov. 8, 2023
(86) PCT No.: PCT/IN2023/051033

§ 371 Date: Jun. 10, 2025

(30) Foreign Application Priority Data
Nov. 14, 2022 (IN) 202221065056

(51) Int. Cl. A61K 9/00; A61K 9/08; A61K 31/7036; A61K 47/02; A61K 47/18; A61P 27/02; A61P 31/04

(52) U.S. Cl. CPC A61K 9/0048; A61K 9/08; A61K 31/7036; A61K 47/02; A61K 47/183; A61K 47/186; A61P 27/02; A61P 31/04

(57) ABSTRACT

The present invention relates to a novel pharmaceutical composition suitable for ophthalmic use comprising antibiotic such as plazomicin or its pharmaceutically acceptable salts and a process to prepare the same for use in the treatment of ocular infections.

### FIELD OF INVENTION

The present invention relates to a novel pharmaceutical composition suitable for ophthalmic use comprising antibiotic and process to prepare the same. More particularly, the present invention relates to a pharmaceutical composition comprising aminoglycoside antibiotics such as plazomicin or its pharmaceutically acceptable salts thereof for the treatment of ocular infections.

### BACKGROUND OF THE INVENTION

Of all the ocular infections caused worldwide, almost up to 74% are believed to be caused by bacteria, followed by viruses, fungus, and parasites. Infection can be mono or poly-microbial and is associated with many factors including contact lenses, trauma, surgery, age, dry eyes, chronic nasolacrimal duct obstruction and previous ocular infections.

Bacteria are generally associated with many types of ocular infections such as conjunctivitis, keratitis, endophthalmitis, blepharitis, orbital cellulitis and dacryocystitis manifestations. The most common bacteria involved in ocular infections are Staphylococcus aureus (S. aureus), Coagulase-negative staphylococci (CoNS), Streptococcus pneumoniae, Corynebacterium spp., Bacillus spp., Nocardia, Pseudomonas aeruginosa, Haemophilus influenzae and Enterobacteriaceae. Among these bacteria, S. aureus and CoNS strains are very important for their high prevalence of infection, while S. aureus is among the most common cause of blepharitis, conjunctivitis, dacryocystitis, keratitis and endophthalmitis.

There are many antibacterial agents which are currently approved to treat ocular infections such as penicillins, fluoroquinolones, tetracyclines, erythromycins, azithromycins, chloramphenicols, aminoglycosides.

Aminoglycoside antibiotics have a broad antibacterial spectrum against gram-positive and gram-negative bacteria, and it is said that their action mechanism is based on inhibition of bacterial protein synthesis. Aminoglycoside antibiotics show long-lasting inhibitory effect on the inhibition of bacterial growth, as they exhibit post-antibiotic effect (PAE) on the inhibition of bacterial growth even after the blood concentration of the aminoglycoside antibiotics is decreased to a concentration of MIC (minimum inhibitory concentration) or less. This long-lasting inhibitory effect on the inhibition of bacterial growth is seen even after a short period of contact with gram-positive and gram-negative bacteria. For this reason, various aminoglycoside antibiotics such as gentamicin, tobramycin, streptomycin, amikacin, arbekacin, and the like have been widely used for the treatment of ocular infections.

However, aminoglycosides like other classes of drugs, are prone to bacterial resistance. Resistance to aminoglycosides may occur based on several mechanisms: (1) enzymatic modification and inactivation of the aminoglycosides, mediated by aminoglycoside acetyltransferases, nucleotidyltransferases, or phosphotransferases and commonly observed across gram-positive and -negative bacteria. Bacterial resistance to aminoglycosides continues to escalate and is widely recognized as a serious health threat. The emergence of ESBL, MBL and pan-drug resistance among P. aeruginosa from ocular infections is an alarming finding which necessitates the earlier detection of both ESBL and MBL production as individual or co-existence in ocular isolates, which may pave the way for appropriate therapy for sight threatening conditions like endophthalmitis.

Thus, there remains a need for the use of novel antibiotic which has excellent activity against Coagulase negative S. aureus, MRSA and retains good activity against gram negative pathogens including Pseudomonas.

Plazomicin is an aminoglycoside that was engineered to overcome aminoglycoside-modifying enzymes (AMEs), the most common aminoglycoside resistance mechanism in the Enterobacteriaceae. Thus, it is an objective of the present invention to provide an ophthalmic composition comprising plazomicin or its pharmaceutically acceptable salts thereof.

### OBJECT OF THE INVENTION

An object of the present invention is to provide an ophthalmic pharmaceutical composition comprising plazomicin or its pharmaceutically acceptable salts thereof.

Another objective of this invention is to develop an ophthalmic composition comprising plazomicin or its pharmaceutically acceptable salts thereof for the treatment of external infections of the eye and its adnexa. The external infections of the eye may be caused by susceptible bacteria like S. aureus (MSSA/MRSA), Coagulase negative S. aureus, S. epidermidis, Klebsiella and Proteus.

It is further an objective of this invention is to develop an ophthalmic composition comprising plazomicin or its pharmaceutically acceptable salts thereof for the treatment of bacterial keratitis. The composition may optionally contain a second antibacterial such as cephalosporin.

### SUMMARY OF THE INVENTION

The present invention relates to a novel pharmaceutical composition suitable for ophthalmic use comprising antibiotic and a process to prepare the same. More particularly, the present invention relates to a pharmaceutical composition comprising aminoglycoside antibiotics such as plazomicin or its pharmaceutically acceptable salts thereof for the treatment of ocular infections.

In one embodiment, the composition of the present invention comprises about 0.01% w/v to about 1.00% w/v plazomicin or its pharmaceutically acceptable salt thereof.

In another embodiment, the composition of the present invention comprises plazomicin sulfate.

In a further embodiment, the composition of the present invention further comprises at least one excipient selected from buffering agent, tonicity adjusting agent, viscosity modifying agent, preservative, chelating agent and pH adjusting agent.

In an embodiment, the composition of the present invention further comprises about 0.01% w/v to about 1.5% w/v buffering agent. In another embodiment, the composition of the present invention further comprises about 0.01% w/v to about 5% w/v buffering agent.

In one more embodiment, the composition of the present invention further comprises about 0.01% w/v to about 1.5% w/v tonicity adjusting agent. In another embodiment, the composition of the present invention further comprises about 0.1% w/v to about 6% w/v tonicity adjusting agent.

In another embodiment, the composition of the present invention further comprises about 0.01% w/v to about 3% w/v preservative. In an embodiment, the composition of the present invention further comprises about 0.00010% w/v to about 1% w/v preservative.

In an embodiment, the composition of the present invention comprises about 0.01% w/v to about 5% w/v viscosity modifying agent.

In one more embodiment, the composition of the present invention comprises about 0.001% w/v to about 1% w/v chelating agent.

In one embodiment, the composition of the present invention further comprises a pH adjusting agent.

In an embodiment, the composition of the present invention is an aqueous composition.

In one another embodiment, the composition of the present invention comprises plazomicin or its pharmaceutically acceptable salt in a solubilized form.

In an embodiment, the present invention relates to an ophthalmic composition for use in ocular infections.

### DETAILED DESCRIPTION OF THE INVENTION

The inventors of this invention have developed an ophthalmic pharmaceutical composition comprising plazomicin or its pharmaceutically acceptable salts thereof and one or more pharmaceutically acceptable excipients.

Plazomicin is chemically known as (2″R,3″R,4″R,5″R)-2″-[(1S,2S,3R,4S,6R)-4-amino-6-[(2′″S)-4′″-amino-2′″-hydroxybutanamido)amino]-3-[(2'S,3′R)-3′-amino-6′-((2-hydroxyethylamino) methyl)-3′,4′-dihydro-2H-pyran-2′-yloxy]-2-hydroxycyclohexyloxy]-5″-methyl-4″-(methylamino)tetrahydro-2H-pyran-3″,5″-diol. The molecular formula is C25H48N6O10 and the molecular weight is 592.69 g/mol. The chemical structure of plazomicin is as given below:

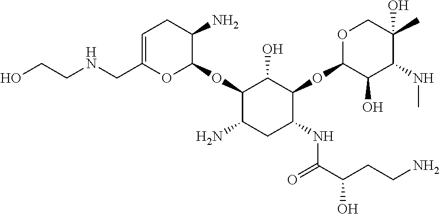 [SMILES: CN[C@@H]1[C@@H](O)[C@@H](O[C@@H]2[C@@H](O)[C@H](O[C@H]3OC(CNCCO)=CC[C@H]3N)[C@@H](N)C[C@H]2NC(=O)[C@@H](O)CCN)OC[C@]1(C)O]

Suitable salts of plazomicin include, but are not limited to sulfate, hydrochloric, hydrobromic, hydroiodic, methylsulfonic, perchloric, sulfuric, nitric, phosphoric, acetic, propionic, glycolic, lactic pyruvic, malonic, succinic, maleic, fumaric, maleic, tartaric, citric, benzoic, carbonic cinnamic, mandelic, methanesulfonic, ethanesulfonic, benezenesulfonic, hydroxyethanesulfonic, p-toluene sulfonic, cyclohexanesulfamic, salicyclic, p-aminosalicylic, 2-phenoxybenzoic, and 2-acetoxy benzoic acid. In a preferred embodiment, the ophthalmic composition contains plazomicin sulfate.

Plazomicin has similar activity to gentamicin (and greater activity than amikacin) against MRSA, MSSA. Plazomicin has potent activity against species of Staphylococcus. Against Gram-negative enteric bacteria, plazomicin demonstrates more potent in vitro activity than amikacin, gentamicin and tobramycin. The inventors of the present invention have intensively studied on the above problems, resulting in finding that an ophthalmic composition containing plazomicin or its pharmaceutically acceptable salts thereof are able to treat the ocular infection, especially the ones where there is a development of bacterial resistance.

Thus, one aspect of the invention is to formulate an ophthalmic pharmaceutical composition comprising plazomicin or its pharmaceutically acceptable salts thereof and one or more pharmaceutically acceptable excipients.

The ophthalmic compositions of the present invention are typically administered to the affected ophthalmic tissues by topically applying one to four drops of a sterile solution or suspension, or a comparable amount of an ointment, gel or other solid or semisolid composition, one to four times per day. However, the compositions may also be formulated as irrigating solutions that are applied to the affected ophthalmic tissues during surgical procedures.

The pharmaceutical ophthalmic compositions, according to the present invention may comprise one or more pharmaceutically acceptable excipients suitable for formulating the same which include, but are not limited to, suspending agents, pH adjusters, tonicity adjusting agents, emulsifiers or dispersing agents, surfactants, solubilizers, buffering agents, preservatives, chelating agents, wetting agents, viscosity modifying agents, antioxidants, gelling agents, stabilizing agents and mixtures thereof.

Ophthalmic pharmaceutical products are typically packaged in multidose form. Preservatives are thus required to prevent microbial contamination during use. Suitable preservatives include but are not limited to quaternary ammonium derivatives, benzalkonium chloride, benzylammonium chloride, cetylmethyl ammonium bromide, stabilized oxychloride complex, quaternium chloride, sodium perborate, benzododecinium bromide, zinc chloride, cetylpyridinium chloride, benzethonium chloride, chlorbutanol; chlorhexidine gluconate, chlorhexidine acetate, organomercury compounds (Thimerosal, phenylmercury acetate, phenylmercury nitrate), methyl and propyl p-hydroxy-benzoates, betaphenylethyl alcohol, benzyl alcohol, phenylethyl alcohol, phenoxyethanol, stabilized oxychloro complex (SOC), sofzia, and sodium perborate and such other agents known to those skilled in the art and mixtures thereof. Typically, such preservatives are employed at a level of from 0.001% to 1.0% by weight. In one embodiment, the composition of the present invention comprises preservatives in an amount of about 0.01% w/v to about 3.00% w/v. In another embodiment, the composition of the present invention comprises preservatives in an amount of about 0.00010% w/v to about 1.00% w/v. In a preferred embodiment, the composition of the present invention comprises about 0.02% w/v benzalkonium chloride.

The ophthalmic pharmaceutical composition of the present invention may optionally comprise one or more suspending agents. Suitable suspending agents can be selected from but are not limited to water soluble/water-swellable polymers such as cellulosic polymers i.e. methyl cellulose, hydroxyl propyl methyl cellulose, hydroxy propyl cellulose, hydroxy ethyl cellulose, carboxy methyl cellulose, polyvinyl pyrrolidine, polyvinyl alcohol, carbopol, carbophil and one or more water-insoluble polymers such as cross-linked carboxyl-containing polymers, ethyl cellulose, and mixtures thereof. Other suspending agents which may be used include, but are not limited to, acacia, agar, alginic acid, sodium alginate, bentonite, carrageenan, gelatin, tragacanth, xanthan gum, and derivatives thereof. Such agents can be used in the range of 0.005% to 1.5% w/v or as deemed fit for the composition.

Suitable surfactants or wetting agents that may be incorporated in the pharmaceutical ophthalmic composition of the present invention may comprise one or more, but are not limited to, Polyoxyethylene fatty acid esters such as polysorbate 20, polysorbate 40, polysorbate 60, polysorbate 80, polysorbate 65, polysorbate 85, sorbitan fatty acid esters such as Span 20, Span 40, Span 60 Span 80, Span 120; sodium lauryl sulfate; polyethoxylated castor oil; polyethoxylated hydrogenated castor oil, sodium dodecyl sulfate, Lauryl dimethyl amine oxide, Docusate sodium, Cetyl trimethyl ammonium bromide, Polyethoxylated alcohols, Octoxynol, N,N-dimethyldodecylamine-Noxide, Polyoxyl 10 lauryl ether, Hexadecyltrimethylammonium bromide, polyoxyethylene surfactant (Brij), Bile salts (such as but not limited to sodium deoxycholate, sodium cholate), Polyoxyl castor oil, Nonylphenol ethoxylate, Cyclodextrins, Lecithin, Methylbenzethonium chloride. Carboxylates, Sulphonates, Petroleum sulphonates, alkylbenzenesulphonates, Naphthalenesulphonates, Olefin sulphonates, Alkyl sulphates, Sulphates, Sulphated natural oils & fats, Sulphated esters, Sulphated alkanolamides, Alkylphenols, Ethoxylated aliphatic alcohol, carboxylic esters, Polyethylene glycol esters, Anhydrosorbitol ester & it's ethoxylated derivatives, Glycol esters of fatty acids, Carboxylic amides, Monoalkanolamine condensates, Polyoxyethylene fatty acid amides, Quaternary ammonium salts, Amines with amide linkages, Polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethylglycine sodium salt, polyoxyethylene/polyoxypropylene surfactants such as Pluronic F-68, F-84, P-103, poloxamines such as Tetronic® 1508, Tetronic® 908, octoxynol 40, tyloxapol, Cremophor and such other surfactants are well known in the art and mixtures thereof. Surfactants can be used in the range of 0.001% w/v to 3.0% w/v.

The composition may further comprise stabilizing agents such as but not limited to phosphonates, such as those sold under the name “DEQUEST” (a trademark of Monsanto); disodium edetate; sodium thiosulfate; and sodium stannate.

The composition of the present invention may further comprise one or more buffers or pH adjusting acids, bases or buffering agents to maintain the pH between 4.5-8 and more preferably between 6.5 to 7.5. Examples of acids include acetic, boric, citric, lactic, phosphoric, hydrochloric and the like and examples of bases include sodium hydroxide, sodium phosphate, sodium borate, sodium citrate, sodium acetate, sodium lactate, THAM (trishydroxymethylaminomethane), tromethamine and the like. Salts and buffers include citrate/dextrose, Sodium Dihydrogen Phosphate Dihydrate, sodium bicarbonate, ammonium chloride or mixtures of the aforementioned acids and bases. In one embodiment, the composition of the present invention comprises about 0.01% w/v to about 1.5% w/v of a buffering agent. In one more embodiment, the composition of the present invention comprises about 0.01% w/v to about 5% w/v of a buffering agent. In a preferred embodiment, the composition of the present invention comprises about 0.3% w/v of boric acid.

In one embodiment, the composition of the present invention may have a pH between about 6 to about 7. In a preferred embodiment, the pH of the composition of the present invention may be between about 6.5 to about 7.

Osmotic/tonicity adjusting agents, that may be used, comprise of sodium chloride, potassium chloride, zinc chloride, calcium chloride and mixtures thereof. Other isotonicity-adjusting agents may also include, but are not limited to, mannitol, anhydrous glucose, glycerol, sorbitol, trehalose, boric acid, citric acid, sodium tartrate, sodium phosphate, potassium phosphate, sodium chloride, glycerin, propylene glycol or other inorganic or organic solutes, dextrose or mixtures thereof. In one embodiment, the composition of the present invention comprise a tonicity adjusting agent in an amount between about 0.01% w/v to about 1.5% w/v. In one more embodiment, the composition of the present invention comprise about 0.10% w/v to about 6% w/v of a tonicity adjusting agent. In a preferred embodiment, the composition of the present invention comprises about 0.73% w/v sodium chloride.

The composition may also contain complexing/chelating agents such as edetic acid (EDTA) or one of the known salts thereof, e.g., sodium EDTA or disodium EDTA dihydrate (sodium edetate), trisodium edetate, malic acid, sodium citrate, condensed sodium phosphate in the concentrations of 0.001% to 1% w/v. In one embodiment, the composition of the present invention comprises about 0.1% w/v EDTA.

The composition may also contain viscosity modifying agents to provide the compositions of the invention with viscosities greater than the viscosity of simple aqueous solutions to increase ocular absorption of the active compounds by the target tissues or increase the retention time in the eye. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents know to those skilled in the art. Such agents can be used in the concentration from 0.01% to 2% w/v. In one embodiment, the composition of the present invention comprises about 0.01% w/v to about 5% of a viscosity modifying agent.

The composition may further comprise sterile water for injection as vehicle. Other aqueous and non-aqueous vehicles may be used as a vehicle for the present ophthalmic composition.

It will be understood, however, that the specific dose level and frequency of dosage according to the invention for any particular patient may be varied and will depend upon a variety of factors including the activity of the specific compound employed, the metabolic stability and length of action of that compound, the age, body weight, general health, sex, diet, mode and time of administration, rate of excretion, the severity of the particular condition, and the host undergoing therapy.

In one embodiment, the composition of the present invention comprises about 0.01% w/v to about 1.00% w/v plazomicin or its pharmaceutically acceptable salt thereof.

In another preferred embodiment, the ophthalmic pharmaceutical composition comprises plazomicin or its pharmaceutically acceptable salts thereof in an amount from about 0.1% w/v to about 5% w/v of the composition. In a more preferred embodiment, the composition comprises plazomicin or its pharmaceutically acceptable salts thereof in an amount from about 0.1% w/v to about 2% w/v of the composition. In another preferred embodiment the composition comprises plazomicin or its pharmaceutically acceptable salts thereof in an amount from about 0.1% w/v to about 0.5% w/v of the composition. In one embodiment, the composition comprises about 0.1% w/v, about 0.2% w/v, about 0.3% w/v, about 0.4% w/v or about 0.5% w/v plazomicin or its pharmaceutically acceptable salts thereof.

According to another embodiment of the present invention, the pharmaceutical composition of the present invention may comprise the actives in micronized form. The actives in micronized form may be obtained by any of the process such as but not limited to ball milling, jet milling, sonication, homogenization and solvent precipitation. According to still another embodiment of the present invention, the pharmaceutical composition of the present invention may also comprise the actives in nanosized form. The nanoparticles of the present invention can be obtained by any of the process such as but not limited to milling, precipitation, homogenization, spray-freeze drying, supercritical fluid technology, double emulsion/solvent evaporation, PRINT, thermal condensation, ultrasonication and spray drying.

According to another embodiment of the present invention the process of milling comprises dispersing drug particles in a liquid dispersion medium in which the drug is poorly soluble, followed by applying mechanical means in the presence of grinding media to reduce the particle size of drug to the desired effective average particle size.

In one embodiment, the composition of the present invention comprises about 0.01% w/v to about 1.00% w/v plazomicin or its pharmaceutically acceptable salt thereof.

In another embodiment, the composition of the present invention comprises plazomicin sulfate.

In a further embodiment, the composition of the present invention further comprises at least one excipient selected from buffering agent, tonicity adjusting agent, viscosity modifying agent, preservative, chelating agent and pH adjusting agent.

In an embodiment, the composition of the present invention further comprises about 0.01% w/v to about 1.5% w/v buffering agent. In another embodiment, the composition of the present invention further comprises about 0.01% w/v to about 5% w/v buffering agent.

In one more embodiment, the composition of the present invention further comprises about 0.01% w/v to about 1.5% w/v tonicity adjusting agent. In another embodiment, the composition of the present invention further comprises about 0.1% w/v to about 6% w/v tonicity adjusting agent.

In another embodiment, the composition of the present invention further comprises about 0.01% w/v to about 3% w/v preservative. In another embodiment, the composition of the present invention further comprises about 0.00010% w/v to about 1% w/v preservative.

In an embodiment, the composition of the present invention comprises about 0.01% w/v to about 5% w/v viscosity modifying agent.

In one more embodiment, the composition of the present invention comprises about 0.001% w/v to about 1% w/v chelating agent.

In one embodiment, the composition of the present invention further comprises a pH adjusting agent.

In an embodiment, the composition of the present invention is an aqueous composition.

In one another embodiment, the composition of the present invention comprises plazomicin or its pharmaceutically acceptable salt in a solubilized form.

In an embodiment, the ophthalmic composition comprises about 0.01% w/v to about 1.00% w/v plazomicin or its pharmaceutically acceptable salt thereof, about 0.01% w/v to about 1.50% w/v of buffering agent, about 0.01% w/v to about 1.50% w/v of tonicity adjusting agent, and water.

In another embodiment, the ophthalmic composition comprises about 0.01% w/v to about 1.00% w/v plazomicin or its pharmaceutically acceptable salt thereof, about 0.01% w/v to about 1.50% w/v boric acid, about 0.01% w/v to about 1.50% w/v sodium chloride, and water.

In a further embodiment, the ophthalmic composition comprises about 0.01% w/v to about 1.00% w/v plazomicin or its pharmaceutically acceptable salt thereof, about 0.01% w/v to about 3.00% w/v preservative, about 0.01% w/v to about 1% w/v of buffering agent, about 0.01% w/v to about 1.50% w/v of tonicity adjusting agent, about 0.01% w/v to about 5% w/v of viscosity adjusting agent and water.

In an embodiment, the ophthalmic composition comprises about 0.01% w/v to about 1.00% w/v plazomicin or its pharmaceutically acceptable salt thereof, about 0.01% w/v to about 3.00% w/v benzalkonium chloride, about 0.01% w/v to about 1% w/v citric acid and disodium citrate, about 0.01% w/v to about 1.50% w/v sodium chloride, about 0.01% w/v to about 5% w/v polyvinyl alcohol and water.

In one more embodiment, the ophthalmic composition comprises about 0.01% w/v to about 1.00% w/v plazomicin or its pharmaceutically acceptable salt thereof, about 0.01% w/v to about 3% w/v preservative, about 0.01% w/v to about 1.50% w/v of tonicity adjusting agent, about 0.01% w/v to about 1.50% w/v of buffering agent, about 0.01% w/v to about 1% w/v of viscosity adjusting agent and water.

In one another embodiment, the ophthalmic composition comprises about 0.01% w/v to about 1.00% w/v plazomicin or its pharmaceutically acceptable salt thereof, about 0.01% w/v to about 3% w/v benzalkonium chloride, about 0.01% w/v to about 1.50% w/v sodium chloride, about 0.01% w/v to about 1.50% w/v boric acid, about 0.01% w/v to about 1% w/v hydroxy propyl guar gum and water.

In another embodiment, the ophthalmic composition comprises about 0.01% w/v to about 1.00% w/v plazomicin or its pharmaceutically acceptable salt thereof, about 0.00010% w/v to about 1% w/v preservative, about 0.01% w/v to about 1.50% w/v of tonicity adjusting agent, about 0.01% w/v to about 1% w/v of buffering agent, about 0.01% w/v to about 5% w/v of viscosity adjusting agent and water.

In a further embodiment, the ophthalmic composition comprises about 0.01% w/v to about 1.00% w/v plazomicin or its pharmaceutically acceptable salt thereof, about 0.00010% w/v to about 1% w/v stabilized oxychloride complex, about 0.01% w/v to about 1.50% w/v sodium chloride, about 0.01% w/v to about 1% w/v citric acid and disodium citrate, about 0.01% w/v to about 5% w/v polyvinyl alcohol and water.

In one embodiment, the ophthalmic composition comprises about 0.01% w/v to about 1.00% w/v plazomicin or its pharmaceutically acceptable salt thereof, about 0.01% w/v to about 3% w/v preservative, about 0.01% w/v to about 1.50% w/v of tonicity adjusting agent, about 0.01% w/v to about 1% w/v of buffering agent, about 0.01% w/v to about 5% w/v of viscosity adjusting agent and water.

In one more embodiment, the ophthalmic composition comprises about 0.01% w/v to about 1.00% w/v plazomicin or its pharmaceutically acceptable salt thereof, about 0.01% w/v to about 3% w/v quaternium chloride, about 0.01% w/v to about 1.50% w/v sodium chloride, about 0.01% w/v to about 1% w/v citric acid and disodium citrate, about 0.01% w/v to about 5% w/v polyvinyl alcohol and water.

In an embodiment, the ophthalmic composition comprises about 0.01% w/v to about 1.00% w/v plazomicin or its pharmaceutically acceptable salt thereof, about 0.01% w/v to about 1% w/v preservative, about 0.01% w/v to about 1.50% w/v of tonicity adjusting agent, about 0.01% w/v to about 1% w/v of buffering agent, about 0.01% w/v to about 5% w/v of viscosity adjusting agent and water.

In another embodiment, the ophthalmic composition comprises about 0.01% w/v to about 1.00% w/v plazomicin or its pharmaceutically acceptable salt thereof, about 0.01% w/v to about 1% w/v sodium perborate, about 0.01% w/v to about 1.50% w/v sodium chloride, about 0.01% w/v to about 1% w/v citric acid and disodium citrate, about 0.01% w/v to about 5% w/v polyvinyl alcohol and water.

In one another embodiment, the ophthalmic composition comprises about 0.01% w/v to about 1.00% w/v plazomicin or its pharmaceutically acceptable salt thereof, about 0.01% w/v to about 3% w/v preservative, about 0.01% w/v to about 1.50% w/v of tonicity adjusting agent, about 0.01% w/v to about 1% w/v of buffering agent, about 0.01% w/v to about 5% w/v of viscosity adjusting agent and water.

In one embodiment, the ophthalmic composition comprises about 0.01% w/v to about 1.00% w/v plazomicin or its pharmaceutically acceptable salt thereof, about 0.01% w/v to about 3% w/v benzododecinium bromide, about 0.01% w/v to about 1.50% w/v sodium chloride, about 0.01% w/v to about 1% w/v citric acid and disodium citrate, about 0.010% w/v to about 5% w/v polyvinyl alcohol and water.

In a further embodiment, the ophthalmic composition comprises about 0.01% w/v to about 1.00% w/v plazomicin or its pharmaceutically acceptable salt thereof, about 0.0010% w/v to about 0.1% w/v preservative, about 0.01% w/v to about 4% w/v of tonicity adjusting agent, about 0.01% w/v to about 1.5% w/v of buffering agent and water.

In an embodiment, the ophthalmic composition comprises about 0.01% w/v to about 1.00% w/v plazomicin or its pharmaceutically acceptable salt thereof, about 0.0010% w/v to about 0.1% w/v zinc chloride, about 0.01% w/v to about 2% w/v propylene glycol, about 0.01% w/v to about 1.5% w/v sodium chloride about 0.01% w/v to about 1.5% w/v boric acid and water.

In one embodiment, the ophthalmic composition comprises about 0.01% w/v to about 1.00% w/v plazomicin or its pharmaceutically acceptable salt thereof, about 0.0010% w/v to about 0.1% w/v preservative, about 0.01% w/v to about 10% w/v of tonicity adjusting agent, about 0.01% w/v to about 1.5% w/v of buffering agent and water.

In one another embodiment, the ophthalmic composition comprises about 0.01% w/v to about 1.00% w/v plazomicin or its pharmaceutically acceptable salt thereof, about 0.0010% w/v to about 0.1% w/v zinc chloride, about 0.01% w/v to about 2% w/v of propylene glycol, about 0.1% w/v to about 6% w/v mannitol, about 0.01% w/v to about 1.5% w/v of boric acid and water.

In an embodiment, the ophthalmic composition comprises about 0.01% w/v to about 1.00% w/v plazomicin or its pharmaceutically acceptable salt thereof, about 0.10% w/v to about 6% w/v of tonicity adjusting agent, about 0.01% w/v to about 2.5% w/v of buffering agent and water.

In another embodiment, the ophthalmic composition comprises about 0.01% w/v to about 1.00% w/v plazomicin or its pharmaceutically acceptable salt thereof, about 0.1% w/v to about 6% w/v of mannitol, about 0.01% w/v to about 2.5% w/v of dibasic sodium phosphate and water.

In another embodiment, the ophthalmic composition comprises about 0.01% w/v to about 1.00% w/v plazomicin or its pharmaceutically acceptable salt thereof, about 0.01% w/v to about 1.5% w/v of tonicity adjusting agent, about 0.01% w/v to about 2.5% w/v of buffering agent and water.

In an embodiment, the ophthalmic composition comprises about 0.01% w/v to about 1.00% w/v plazomicin or its pharmaceutically acceptable salt thereof, about 0.01% w/v to about 1.5% w/v of sodium chloride, about 0.01% w/v to about 2.5% w/v of dibasic sodium phosphate and water.

In a further embodiment, the ophthalmic composition comprises about 0.01% w/v to about 1.00% w/v plazomicin or its pharmaceutically acceptable salt thereof, about 0.1% w/v to about 6% w/v of tonicity adjusting agent, about 0.01% w/v to about 5% w/v of buffering agent and water.

In another embodiment, the ophthalmic composition comprises about 0.01% w/v to about 1.00% w/v plazomicin or its pharmaceutically acceptable salt thereof, about 0.1% w/v to about 6% w/v of mannitol, about 0.01% w/v to about 2.5% w/v of dibasic sodium phosphate, about 0.01% w/v to about 2.5% w/v of monobasic sodium phosphate and water.

In an embodiment, the ophthalmic composition comprises about 0.01% w/v to about 1.00% w/v plazomicin or its pharmaceutically acceptable salt thereof, about 0.01% w/v to about 1.5% w/v of tonicity adjusting agent, about 0.01% w/v to about 5% w/v of buffering agent and water.

In a further embodiment, the ophthalmic composition comprises about 0.01% w/v to about 1.00% w/v plazomicin or its pharmaceutically acceptable salt thereof, about 0.01% w/v to about 1.5% w/v of sodium chloride, about 0.01% w/v to about 2.5% w/v of dibasic sodium phosphate, about 0.01% w/v to about 2.5% w/v of monobasic sodium phosphate and water.

In an embodiment, the ophthalmic composition comprises about 0.01% w/v to about 1.00% w/v plazomicin or its pharmaceutically acceptable salt thereof, about 0.01% w/v to about 1.5% w/v of tonicity adjusting agent, about 0.01% w/v to about 5% w/v of buffering agent, about 0.01% w/v to about 3% w/v of preservative, about 0.001% w/v to about 1% w/v of chelating agent and water.

In another embodiment, the ophthalmic composition comprises about 0.01% w/v to about 1.00% w/v plazomicin or its pharmaceutically acceptable salt thereof, about 0.73% w/v of tonicity adjusting agent, about 0.3% w/v of buffering agent, about 0.02% w/v of preservative, about 0.1% w/v of chelating agent and water.

In a further embodiment, the ophthalmic composition comprises about 0.01% w/v to about 1.00% w/v plazomicin or its pharmaceutically acceptable salt thereof, about 0.01% w/v to about 1.5% w/v of sodium chloride, about 0.01% w/v to about 5% w/v of boric acid, about 0.01% w/v to about 3% w/v of benzalkonium chloride, about 0.001% w/v to about 1% w/v of EDTA and water.

In one more embodiment, the ophthalmic composition comprises about 0.01% w/v to about 1.00% w/v plazomicin or its pharmaceutically acceptable salt thereof, about 0.73% w/v of sodium chloride, about 0.3% w/v of boric acid, about 0.02% w/v of benzalkonium chloride, about 0.1% w/v of EDTA and water.

In an embodiment, the present invention relates to an ophthalmic composition for use in ocular infections.

According to an embodiment of the present invention, the invention also relates to a process of preparing ophthalmic pharmaceutical composition which comprises (i) autoclaving a slurry comprising the active ingredients and optionally other excipients; (ii) preparing a solution comprising other pharmaceutically acceptable excipients and autoclaving the same; (iii) aseptically adding the drug slurry to the excipient solution and (iv) finally making up the volume with vehicle.

According to preferred embodiment of the present invention, the invention also relates to a process of preparing ophthalmic pharmaceutical composition which comprises (i) milling and autoclaving a slurry comprising the active ingredient, surfactant and suspending agent; (ii) preparing a solution comprising other pharmaceutically acceptable excipients and autoclaving the same; (iii) aseptically adding the preservative to the excipient solution; (iv) aseptically adding the drug slurry to the excipient solution and (v) finally making up the volume with vehicle.

According to another embodiment of the present invention, the invention also relates to a process of preparing ophthalmic pharmaceutical composition which comprises (i) milling and autoclaving a slurry comprising the active ingredient, surfactant and suspending agent; (ii) preparing a solution comprising other pharmaceutically acceptable excipients, and preservative and autoclaving the same; (iii) aseptically adding the drug slurry to the excipient and preservative solution and (iv) finally making up the volume with vehicle.

The present invention also provides a method for the treatment and/or prevention of ophthalmic infections, which method comprises the administration of a therapeutically effective amount of a pharmaceutical composition according to the present invention.

The composition of the present invention may be used topically in the treatment of ocular infections associated with inflammation such as conjunctivitis, dacryocystitis, hordeolum, keratitis, blepharitis and corneal ulcers thereof. The composition of the present invention may also be used prophylactically in connection with various ophthalmic surgical procedures that create a risk of infection.

The following examples are for the purpose of illustration of the invention only and are not intended in any way to limit the scope of the present invention:

### Example 1

Quantity in w/v

| Components | A | B | C | D | E | F |
|---|---|---|---|---|---|---|
| Plazomicin | 0.01%- | 0.01%- | 0.01%- | 0.01%- | 0.01%- | 0.01%- |
| | 1.00% | 1.00% | 1.00% | 1.00% | 1.00% | 1.00% |
| Benzalkonium | | 0.01%- | 0.01%- | | | |
| Chloride | | 3.00% | 3.00% | | | |
| Boric Acid | 0.01%- | | 0.01%- | | | |
| | 1.50% | | 1.50% | | | |
| Stabilized | | | | 0.00010%- | | |
| Oxychloride | | | | 1.00% | | |
| Complex (SOC) | | | | | | |
| Quaternium Chloride | | | | | 0.01%- | |
| | | | | | 3.00% | |
| Sodium Perborate | | | | | | 0.01%- |
| | | | | | | 1.00% |
| Citric acid | | 0.01%- | | 0.01%- | 0.01%- | 0.01%- |
| | | 1.00% | | 1.00% | 1.00% | 1.00% |
| Sodium Citrate, | | 0.01%- | | 0.01%- | 0.01%- | 0.01%- |
| Dihydrate | | 1.00% | | 1.00% | 1.00% | 1.00% |
| Sodium Chloride | 0.01%- | 0.01%- | 0.01%- | 0.01%- | 0.01%- | 0.01%- |
| | 1.50% | 1.50% | 1.50% | 1.50% | 1.50% | 1.50% |
| Polyvinyl alcohol | | 0.01%- | | 0.01%- | 0.01%- | 0.01%- |
| | | 5.00% | | 5.00% | 5.00% | 5.00% |
| Hydroxy Propyl Guar | | | 0.01%- | | | |
| Gum | | | 1.00% | | | |

| | |
|---|---|
| Sodium Hydroxide | q.s pH 6.5 to 7 |
| Hydrochloric Acid | q.s pH 6.5 to 7 |
| Water for Injection | q.s 100 |

| | Quantity in w/v | | | | | | |
|---|---|---|---|---|---|---|---|
| Components | G | H | I | J | K | L | M |
| Plazomicin | 0.01%- | 0.01%- | 0.01%- | 0.01%- | 0.01%- | 0.01%- | 0.01%- |
| | 1.00% | 1.00% | 1.00% | 1.00% | 1.00% | 1.00% | 1.00% |
| Sodium | | | | 0.01%- | 0.01%- | 0.01%- | 0.01%- |
| Phosphate, | | | | 2.50% | 2.50% | 2.50% | 2.50% |
| Dibasic | | | | | | | |
| Sodium | | | | | | 0.01%- | 0.01%- |
| Phosphate, | | | | | | 2.50% | 2.50% |
| Monobasic | | | | | | | |
| Benzododecinium | 0.01%- | | | | | | |
| Bromide | 3.00% | | | | | | |
| Zinc Chloride | | 0.0010%- | 0.0010%- | | | | |
| | | 0.1% | 0.1% | | | | |
| Propylene | | 0.01%- | 0.01%- | | | | |
| glycol | | 2.00% | 2.00% | | | | |
| Boric Acid | | 0.01%- | 0.01%- | | | | |
| | | 1.50% | 1.50% | | | | |
| Citric acid | 0.01%- | | | | | | |
| | 1.00% | | | | | | |
| Sodium | 0.01%- | | | | | | |
| Citrate, | 1.00% | | | | | | |
| Dihydrate | | | | | | | |
| Sodium | 0.01%- | 0.01%- | | | 0.01%- | | 0.01%- |
| Chloride | 1.50% | 1.50% | | | 1.50% | | 1.50% |
| Mannitol | | | 0.10%- | 0.10%- | | 0.10%- | |
| | | | 6.00% | 6.00% | | 6.00% | |
| Polyvinyl | 0.01%- | | | | | | |
| alcohol | 5.00% | | | | | | |
| Sodium | q.s pH 6.5 to 7 | | | | | | |
| Hydroxide | | | | | | | |
| Hydrochloric | q.s pH 6.5 to 7 | | | | | | |
| Acid | | | | | | | |
| Water for | q.s 100 | | | | | | |
| Injection | | | | | | | |

| Sr. | | Qty./ ml | Qty./ ml | |
|---|---|---|---|---|
| No. | Ingredients | (% w/v) | (mg/ mL) | Function |
| 1 | Plazomicin | 0.01-1.00 | 0.10-10.00 | Active |
| 2 | Benzalkonium Chloride | 0.02 | 0.20 | Preservative |
| 3 | Boric Acid | 0.30 | 3.00 | Buffering |
| | | | | Agent |
| 4 | Sodium Chloride | 0.73 | 7.30 | Tonicity Agent |
| 5 | EDTA | 0.10 | 1.00 | Chelating agent |
| 6 | Sodium Hydroxide | q.s. pH 6.5 to 7.0 | | pH adjusting |
| 7 | Hydrochloric Acid | | | agent |
| 8 | Water for Injection | q.s 100% | q.s. 1 ml | Vehicle |

### Manufacturing Process:

1. Dissolve benzalkonium chloride in water for injection previously sparged with nitrogen.
2. Dissolve boric acid in solution of step 1 under stirring.
3. Dissolve sodium chloride and EDTA in solution of step 2 under stirring.
4. Add and dissolve plazomicin under stirring to solution of step 3.
5. Adjust pH of solution of step 4 with sodium hydroxide/hydrochloric acid to 6.5 to 7.
6. Adjust volume of solution of step 5 with water for injection.

### Example 3: In Vitro “Proof of Concept” Study of Effects of Plazomicin on Isolates from Bacterial Keratitis and/or Conjunctivitis

Isolates from patients diagnosed clinically with microbial keratitis were tested for antibacterial susceptibility against plazomicin agents using reference broth microdilution methods.

a. Preparation of Muller Hinton Broth

21 grams of Muller Hinton broth powder was suspended in 1000 ml of distilled water.

b. Bacterial Isolates

Pure cultures of bacterial isolates were prepared in Blood Agar/Mueller Hinton agar from cryovials.

c. Inoculum Preparation:

A standardized inoculum of 0.5 McFarland was prepared using direct colony suspension method.

3-5 well isolated colonies were taken from 18-24 hour culture plate and suspended into 2 ml of sterile saline/Muller Hinton broth.

A homogenous suspension was made, and the turbidity was adjusted to 0.5McFarland turbidity standard (approximately 1*10⁶⁻⁸ CFU/ml) using the Densitometer.

A 0.1 ml of standardized bacterial inoculum was added to 9.9 ml of sterile saline solution. (i.e., a 1:100 dilution).

d. Microbroth Dilution Method

A 96 well round bottomed plate was used for the Microbroth dilution assay.

A 100 μl of Muller Hinton broth containing drug concentrations from 0.125 mcg/ml to 64 mcg/ml (0.125 mcg/ml, 0.25 mcg/ml, 0.5 mcg/ml, 1 mcg/ml, 2 mcg/ml, 4 mcg/ml, 8 mcg/ml, 16 mcg/ml, 32 mcg/ml, and 64 mcg/ml) were added to the 96 well.

To this a 100 μl of the diluted bacterial inoculum (1:100) was added to each well with varying concentration of drugs. Each well contained 5*10⁵ CFU/ml bacteria.

Growth control (Respective organism+Muller Hinton broth) and sterility control (Sterile Muller Hinton broth) were added to the 96 well plate.

The plate was incubated for 18-24 hours to read the Minimum Inhibitory Concentration (MIC).

Gram positive isolates evaluated:
1. Methicillin susceptible Staphylococcus aureus (MSSA)
2. Methicillin resistant Staphylococcus aureus (MRSA)
3. Staphylococcus epidermidis
4. Staphylococcus hominis
5. Staphylococcus haemolyticus
6. Streptococcus pneumoniae
7. Corynebacterium

### Gram Negative Isolates Evaluated:

1. Pseudomonas
2. Klebsiella pneumoniae
3. Klebsiella pneumoniae (ESBLs)
4. Moraxella lacunata
5. Haemophilus influenzae
6. Proteus
7. Serratia
8. Enterobacter
   e. Results:

Plazomicin was active against MSSA isolates and 67.3% were susceptible (MIC_{50/90}, 2/8 mcg/mL). Further 60% of MRSA isolates were susceptible (MIC_{50/90}, 2/8 mcg/mL). Plazomicin showed activity for 50% of Pseudomonas spp. (MIC_{50/90}, 4/16 mcg/mL). Majority of the Staphylococcus spp. (90.10%) were inhibited by plazomicin at ≤8 mcg/mL, while 73.5% were inhibited at ≤4 mcg/mL and 46% were inhibited at ≤2 mcg/mL. Plazomicin showed 100% activity for S. epidermidis, S. hominis, S. haemolyticus, Moraxella lacunata, Enterobacter spp., and Corynebacterium at ≤2 mcg/mL. Plazomicin inhibited 61.5% and 11.5% of the Enterobacterales isolates at ≤2 mcg/mL and ≤4 mcg/mL, respectively.

To conclude, Plazomicin demonstrated very good antibacterial activity. The conjunctival isolates showed 100% susceptibility to plazomicin (≤4 mcg/mL).

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a buffering agent” includes a single buffering agent as well as two or more different buffering agents; reference to a “solvent” refers to a single cosolvent or to combinations of two or more solvents, and the like.

We claim:

1. An ophthalmic composition comprising plazomicin or its pharmaceutically acceptable salt thereof.

2. The composition of claim 1, wherein the composition comprises about 0.01% w/v to about 1.00% w/v plazomicin or its pharmaceutically acceptable salt thereof.

3. The composition of claim 1, wherein the composition comprises plazomicin sulfate.

4. The composition of claim 1, wherein the composition further comprises at least one excipient selected from buffering agent, tonicity adjusting agent, viscosity modifying agent, preservative, chelating agent and pH adjusting agent.

5. The composition of claim 1, wherein the composition further comprises about 0.01% w/v to about 1.5% w/v buffering agent.

6. The composition of claim 1, wherein the composition further comprises about 0.01% w/v to about 5% w/v buffering agent.

7. The composition of claim 1, wherein the composition further comprises about 0.01% w/v to about 1.5% w/v tonicity adjusting agent.

8. The composition of claim 1, wherein the composition further comprises about 0.1% w/v to about 6% w/v tonicity adjusting agent.

9. The composition of claim 1, wherein the composition further comprises about 0.01% w/v to about 3% w/v preservative.

10. The composition of claim 1, wherein the composition further comprises about 0.00010% w/v to about 1% w/v preservative.

11. The composition of claim 1, wherein the composition further comprises about 0.01% w/v to about 5% w/v viscosity modifying agent.

12. The composition of claim 1, wherein the composition further comprises about 0.001% w/v to about 1% w/v chelating agent.

13. The composition of claim 1, wherein the composition further comprises a pH adjusting agent.

14. The composition of claim 1, wherein the composition is an aqueous composition.

15. The composition of claim 1, wherein the plazomicin or its pharmaceutically acceptable salt is in a solubilized form.

16. The composition of claim 1, wherein the composition has a pH between about 6 to about 7.

17. The composition of claim 1 for use in the treatment of ocular infections.
